(19) United States
(12) Patent Application Publication
(10) Pub. No.: US 2026/0268202 A1
(43) Pub. Date: Sep. 10, 2026

(54) COMPUTER-IMPLEMENTED TRAINING METHOD, COMPUTER-IMPLEMENTED PREDICTION METHOD, COMPUTER PROGRAM, COMPUTER-READABLE MEDIUM AND DEVICE

(71) Applicant: Forschungszentrum Jülich GmbH, Jülich (DE)

(72) Inventor: Kaustubh Raosaheb PATIL, Düsseldorf (DE)

(21) Appl. No.: 18/871,721

(22) PCT Filed: Jun. 7, 2023
(86) PCT No.: PCT/EP2023/065239

§ 371 Date: Dec. 4, 2024

(30) Foreign Application Priority Data
Jun. 10, 2022 (DE) 10 2022 114 713.4

(51) Int. Cl. G06N 20/00; G16H 50/20

(52) U.S. Cl. CPC G06N 20/00; G16H 50/20

(57) ABSTRACT

A computer-implemented training method for training machine learning models includes learning data collection with learning data sets. Each learning data set comprises a measurement data set and a target variable. A grouping of the measurement data entries of the measurement data sets is formed into multiple groups. For each group, its own machine learning model is trained and tested using a resampling method and a testing process with another portion of the group sub measurement data sets belonging to the group. The testing process provides predictions for different groups, and group-specific predictions. The group-specific predictions and target variables of at least one other machine learning model and cross-group meta learning model is trained, such that it can provide a cross-group prediction from multiple group-specific predictions associated with different groups. The invention also relates to a computer-implemented prediction method, a computer program, a computer-readable medium, and a device.

The invention relates to a computer-implemented training method for training machine learning models, in which
A1) at least one learning data collection with several learning data sets is provided, wherein each learning data set comprises a measurement data set with several measurement data entries and a target variable assigned to the measurement data set, preferably wherein all learning data sets have the same structure.

The invention further relates to a computer-implemented prediction method for predicting a property using machine learning models, a computer program, a computer-readable medium and a device.

Artificial intelligence and machine learning (ML) are becoming an important tool in the analysis of data, such as image data [Myszczynska et al. 2022]. One example is image data of human brains obtained by magnetic resonance imaging (MRI). However, there are various challenges that make the application of ML methods more difficult. One major challenge, particularly in the clinical field, is the availability of comparatively little data at the respective measurement location, for example the hospital where MRI images are taken. Since large amounts of training data are usually required to build accurate ML models, a common approach is to increase the amount of data by pooling data obtained at different measurement sites. However, multi-location data pooling is also associated with challenges.

On the one hand, harmonization is required because the data from different locations is heterogeneous, in particular because there are systematic differences between the measurement setups used, such as scanners in the case of MRI images, and the data collection parameters [Chen 2021, Mali et al]. Pooling therefore requires additional processing, which is achieved by building data harmonization models.

Furthermore, even if standard precautions, such as pseudonymization of images, are observed, sharing sensitive data across sites can lead to a reduction in patient privacy, as fingerprint-like properties of the data can be used for re-identification [Finn 2015, Larabi 2021]. Various methods, such as defacing of MRI images, are currently used to increase patient privacy. Federated learning (FL) is used to increase data privacy [Kaissis et al. 2020].

The existing solutions have various limitations and disadvantages.

In the case of cross-location data pooling, the harmonization models usually require some prototypical data from the respective measurement location for training. This limits the applicability with regard to new measurement locations for which no prototypical data is available. Harmonization can also be problematic in terms of data protection, especially when raw data is merged. Decentralized learning, e.g. federated learning, can lead to models with lower accuracy. Another disadvantage is when non-interpretable models, such as deep learning-based methods, are used. The lack of interpretability makes it difficult to use for critical decision-making situations, such as those that often exist in the clinical field [Hedderich & Eickhoff 2021].

Based on this, it is a task of the present invention to create a possibility of obtaining particularly accurate predictions with a particularly high degree of privacy or data protection at the same time, also using data from different measurement locations.

In a computer-implemented training method of the type mentioned above, this is solved by the fact that
A2) at least some of the measurement data entries of the measurement data sets of the at least one learning data collection are grouped into a plurality of groups, whereby group sub-measurement data sets are obtained, wherein the group sub-measurement data records of a group each comprise measurement data entries of different measurement data records which correspond to one another,
A3) for each group a separate machine learning model is trained and tested using a resampling method, resampling-based group learning model, wherein the resampling-based method includes that at least one training is performed with a part of the group sub-measurement data sets belonging to the respective group and the associated target variables, and at least one testing is performed with another part of the group sub-measurement data sets belonging to the respective group, and wherein the at least one testing provides predictions for different groups, group-specific predictions,
A4) at least one further machine learning model, cross-group meta-learning model, is trained with the group-specific predictions and target variables of the at least one learning data collection, in particular without using a resampling method, so that it can provide a cross-group prediction from several group-specific predictions assigned to different groups,
A5) optionally for each group a further machine learning model is trained with group sub-measurement data sets belonging to the respective group (and associated target variables in particular without using a resampling method, prediction group learning model, whereby the training is preferably carried out with all group sub-measurement data sets belonging to the respective group and associated target variables.

The learning models trained according to the invention can be used in particular to make at least one prediction for at least one new measurement data set for which no target variable is available. By way of example only, MRI data of the brain of a patient is available and group-specific, in particular cell-specific brain ages and a cross-cell brain age are predicted for this patient. Other possible application examples for the invention can be found in genetics, medical patient data, natural language processing and computer vision.

It should be emphasized that the steps of the training method according to the invention do not necessarily have to be carried out in the aforementioned order. In particular, the training of the non-resampling-based group learning models according to step A5) can also take place earlier, e.g. between step A2) and A3) or simultaneously with step A3).

It is therefore also an object of the invention to provide a computer-implemented prediction method for predicting a property using machine learning models obtained by performing the training method according to the invention, in which
B1) a measurement data set with several measurement data entries is provided,
B2) prediction group learning models trained according to step A5 and/or resampling-based group learning models trained according to step A2 and at least one cross-group meta-learning model trained according to step A4, in particular without using a resampling method, are provided,
B3) the measurement data entries of the measurement data set provided in step B1 are grouped analogously to step A2, whereby a group sub-measurement data set is obtained for each group,
B4) the group sub-measurement data sets are each fed to the associated prediction group learning models or the associated resampling-based group learning models and a group-specific prediction is obtained as output from each of these,
B5) the group-specific predictions are fed to the at least one cross-group meta-learning model trained in particular without using a resampling method and a cross-group prediction is obtained as output from this model.

It should be emphasized that the steps of the prediction method according to the invention do not necessarily have to be carried out in the aforementioned order. For example, the learning models can also be provided after the grouping.

In other words, the present invention provides for a divide-and-conquer approach in which multiple levels of machine learning models are trained. Several levels are also run through in the context of prediction using the trained models. The outputs of one level are used—during training and prediction—as inputs for the next, “higher” level.

According to the invention, the training data is divided into groups and a machine learning model is introduced for each group, particularly at the first, “lowest” level (level 0), and trained and tested using a resampling-based method, in other words group-specific learning models or group learning models. These are trained and tested using a resampling-based method with the learning data.

As part of the grouping, different parts or sections of the measurement data sets are conveniently assigned to different groups. By way of example only, a subdivision into two or more groups is carried out, whereby one group is assigned one part of the measurement data entries of the measurement data sets and the other group or groups are assigned the other part(s) of the measurement data entries. The grouping is such that corresponding parts or areas of the measurement data sets, i.e. corresponding measurement data entries, are each assigned to the same group. For example, a first group comprises the first five measurement data entries of the measurement datasets, another group comprises the next seven measurement data entries of all measurement data sets and so on. If the measurement data sets are given by image data or comprise such data, a group includes in particular the same image section of each measurement data set, i.e. each image or each recording.

The groups can be mutually exclusive, in other words disjoint, or overlap, i.e. include common measurement data entries. The group sub-measurement data records of different groups should differ from each other. The groups can be flat or organized in a structure, such as a hierarchy. It is possible that all measurement data entries are assigned to one or more groups or that this only applies to some of the measurement data entries. It is also possible for only one measurement data entry or several measurement data entries to be assigned to a group. For example, one or more or each group can consist of exactly one measurement data entry.

For example, a grouping predefined by a user or a grouping of the measurement data records derived from the respective learning data collection, in particular the structure of the measurement data records, preferably obtained by clustering, can be carried out.

The resampling method involves dividing the data once or several times into a training and a test part, which can also be referred to as training and test sets. From one part, the training part, the sub-measurement data sets together with the associated target variables are then used to train the respective group learning model so that it can predict (further) target variables in the trained state.

In this way, machine learning models are trained for each group, in other words trained using measurement data sets that are assigned to the respective group. Each sub-measurement data set is part of a measurement data set and is therefore assigned or belongs to it. Each measurement data set is assigned a carry variable, which can also be considered to be assigned to the sub-measurement data set or to belong to the sub-measurement data set.

The sub-data sets of the respective group belonging to the other part, the test part, are then fed into the respective group learning model (without the corresponding, associated target variables) in order to obtain predictions from this. This is also referred to as “out of sample” predictions. Training with one part and testing with another part of the data can be carried out several times in a known manner within the framework of resampling-based modelling. By way of example, the measurement data sets can be divided into two parts of the same or different sizes, trained with one part and predicted with the other in a first run and then swapped in a second run, i.e. trained with the other part and tested with one part. In this way, a number of “out of sample” predictions can be obtained that corresponds to the total number of measurement data sets, although this is by no means mandatory, but only an option. Since the resampling-based modelling according to the invention is done for each group, group-specific predictions are obtained.

At least one meta-model can then be trained using the group-specific predictions and the target variables of the learning data set. After training, the at least one meta-model is able to provide a common, cross-group prediction as output on the basis of several group-specific predictions as input.

Optionally, in step A5, a further machine learning model is trained for each group with group sub-measurement data sets belonging to the respective group and associated target variables in an expedient manner without using a resampling method. These models are also referred to here as prediction group learning models, as they can be used primarily for the prediction method according to the invention. Here, a learning model without using a resampling method also includes learning models that were derived or summarized from previously trained resampling-based learning models. For example, ensemble models from models that were previously trained based on resampling, e.g. by k-fold cross-validation, are also mentioned.

At prediction time, a new instance runs through all levels of the stack in succession, i.e. the trained machine learning models of all levels, and predictions are obtained for each level. The final prediction from the last, highest level and the intermediate predictions from the previous level(s) can be used to interpret and make decisions.

In principle, various types of predictions can be made in the context of the training and prediction method according to the invention, for example predicted values or prediction probabilities or confidence. Accordingly, the prediction target variables may, for example, be predicted values, prediction probabilities or also confidences, or they may comprise such. It should be noted that the term target variable can also be used as an alternative to the term target variable.

Since the models in each level predict the outcome or target, the output of each level is more focused on the target than on any other information contained in the raw data, especially the measurement datasets. In other words, by outputting target predictions or estimates, each level creates a more abstract representation of the data and thus reduces private information. This makes it possible to ensure a high level of privacy, especially when sharing or merging data across measurement locations, which is an important advantage of the invention. The invention makes it possible to share less private data while obtaining more accurate models using data from multiple sites (“cross-site”).

Bootstrapping and cross-validation, such as leave-one-out cross-validation or k-fold cross-validation, are purely exemplary resampling methods that can be used in the context of the invention. Of course, a combination of different resampling methods can also be used, both within a level and across different levels.

The training of machine learning models in the context of the present invention may further comprise the tuning of hyperparameters, whereby this applies to all learning models at all levels.

It has proven to be particularly suitable if two, three or four levels or layers of machine learning models are provided in the context of the present invention. One can also speak of a stack with two, three or four levels. However, it is also by no means excluded that more levels are used. In particular, a number of levels or layers of the stack can be provided that is adapted to the grouping. Levels can also be organized hierarchically. The hierarchical structuring of brain MRI data using the brain parcellation scheme of Yeo et al. (2011) is a purely exemplary example. Here, it would be possible to first use the voxels in Level 1 as a grouping for modelling the target variable based on their brain parcellations. In Level 2, the predictions per brain parcel could be used to model the target variable as a grouping based on their brain networks as defined by Yeo et. al. This is a hierarchical structure in that each network consists of multiple parcels made up of multiple voxels. In this example, not all voxels would be used as input, as this brain parcellation scheme only considers cerebral cortex. Now, all unused voxels could remain unconsidered in the training procedure or be used additionally for further hierarchical or non-hierarchical modelling by adding other parcellation schemes. This means that in a level 4 the predictions can now be used grouped by different parcellation schemes. This includes either different networks or brain parcellations or a mixture of both. This is just one example of the possibilities within the scope of the present invention. Other combinations of hierarchical or non-hierarchical, overlapping or non-overlapping groupings are also possible.

The machine learning models used in the context of the present invention can be of any type. In particular, a user is free to choose which model or models to use for the different groups and levels. In particular, the models can comprise one or more arbitrary machine learning algorithms or be given by them. Examples of machine learning algorithms include decision trees and support vector machines. If inherently interpretable models are selected, the overall solution becomes interpretable as such.

In one embodiment of the training method according to the invention, it is provided that the different learning data sets belong to different patients and the learning data sets comprise medically relevant measurement data sets, in particular measurement data sets obtained by means of medical diagnostic methods, and the target variables relate to or are given by characteristics of the patients, in particular an age and/or a disease state of the patients.

The measurement data sets may, for example, be provided by or comprise image recordings, in particular MRI image recordings, of human brains. The grouping according to the invention can then be carried out in parcels, so that each group corresponds to a specific parcel with specific, suitably connected voxels. The term voxel is composed of “volumetric” and “pixel” and corresponds in a known manner to the 3D equivalent of a pixel. The measurement data entries can then in particular be voxel-by-voxel measurements of MRI data, such as the gray cell volume, whereby the voxels of a specific brain cell belong to each group.

In the specific case of brain MRI in particular, the invention can be used to exploit known regularities in the data in order to initially make location-based predictions, which are followed by final, cross-brain predictions, in other words predictions assigned to the entire brain. One can imagine, for example, an age or gender prediction that can serve as sample cases for regression or classification. These sample cases offer clinical applicability, are well established, are known to offer high accuracy, and their results, including feature significance, are easy to verify. For example, it is expected that the volume of gray matter decreases with age, so that a negative feature weighting can be assumed. In the context of the invention, the lowest level of the stack will build predictive models using a priori defined groupings of brain regions. Following the training phase, the trained group-specific models and the meta-model or meta-models can be used to make predictions for a provided measurement dataset, such as an MRI image of a patient.

In the case of MRI images of patients, such as their brains, the target variables can be given, for example, by the age of the respective patient or a disease status. As part of the prediction process, a patient's age or disease status can be predicted accordingly, for example.

A further embodiment is characterized in that the different learning data sets belong to different persons and the measurement data records are given by or comprise image recordings of at least part of the face and/or at least part of the body of the persons, and the target variables relate to or are given by properties of the persons. The measurement data entries can then each correspond to or be assigned to a pixel. Furthermore, it may be provided that the characteristics of the persons do not relate to or are not given by a disease state of the persons.

In other words, training can also be performed using images of faces or parts of faces and/or the bodies or body parts of persons and characteristics of the persons, such as age and/or gender. Following the training phase, the trained group-specific models and the meta-model or meta-models can be used to make predictions for a provided measurement data set, for example an image of a person's face, e.g. to predict the person's age and/or gender.

With regard to the grouping according to the invention, in this case it can apply, for example, that a grouping is made according to certain areas or parts of the face (e.g. nose, eyes, . . . ) and/or body.

It is also possible that the different learning data sets belong to different sections of the earth's surface and the measurement data sets are given by or comprise image recordings, in particular satellite images, of the earth's surface sections, and the target variables relate to or are given by properties of the earth's surface sections, in particular the presence of certain elements, preferably the presence of fields and/or rivers and/or lakes.

In other words, training can be carried out using satellite images of the earth's surface and certain landscape features associated with the section shown, such as the existence of fields, lakes, rivers, etc. Following the training phase, the trained group-specific models and the meta-model or meta-models can be used to make predictions for a new satellite image with unknown features, such as predicting whether it shows rivers, lakes and/or fields, to name just a few examples of possible features.

With regard to the grouping provided according to the invention, in this case it can apply in particular that grouping takes place according to specific areas or parts of the image recordings. For example, a segmentation (image segmentation) can be carried out, in the context of which regions or areas that are preferably related in terms of content are determined, which comprise neighbouring pixels that fulfil a certain homogeneity criterion. The resulting segments can then each represent a group or correspond to such a group.

As a further example, it may be mentioned that the different learning data sets belong to different persons and the measurement data sets are given by or comprise information about the user behaviour of the persons, in particular on at least one website, and the target variables relate to or are given by information about actions performed by the persons, in particular purchases made by the persons. The actions in question are expediently actions performed on the at least one website, such as purchases of products and/or services offered on the at least one website.

The information about the user behaviour of the persons covered by the measurement data records can include, for example, information about how long the persons spent on a website, in which area or areas of the website the persons moved the mouse pointer and, in particular, how long the mouse pointer remained in the respective areas, and/or in which areas the persons clicked with the mouse pointer.

In other words, training can take place with information about user behaviour, especially on the Internet, and associated specific user actions, such as purchases.

Following the training phase, the trained group-specific models and the meta-model or meta-models can be used to make predictions about actions or probabilities of action, such as purchase probabilities for certain products and/or services, based on data about the user behaviour of at least one other person.

With regard to the grouping, it can then apply, for example, that different areas of the at least one website form different groups or correspond to such groups, in other words a grouping into different website areas or sections takes place.

A further embodiment example is characterized in that the different learning data sets belong to different DNA sequences and/or protein sequences and/or gene expressions and the measurement data sets are given by or comprise information about the DNA sequences and/or protein sequences and/or gene expressions. Expediently, the information then concerns the structure of the DNA sequences and/or protein sequences and/or gene expressions. The information may comprise sequence-based features of DNA sequences and/or protein sequences or gene expression data. The target variables then expediently relate to (other) features of the DNA sequences and/or protein sequences and/or gene expression data, in particular binding sites and/or protein-protein interactions and/or solvent properties thereof, or are given thereby.

Following the training phase, the trained group-specific models and the meta-model or meta-models can then be used, for example, to predict binding sites or protein-protein interactions or solvent properties based on information for a new DNA or protein sequence or gene expression data, in particular based on an associated measurement data set.

With regard to grouping, for example, it is possible to define groups using domain knowledge, such as known gene regions or interaction networks.

The measurement data for the training can come from one or more measurement locations. In the latter case, separate group learning models, in other words measurement location-specific group learning models, can be introduced for each measurement location.

In a preferred further development of the training method according to the invention, it is therefore provided that in step A1 learning data sites are provided from and in particular at different measurement locations, the learning data collections each comprising several learning data sets with measurement data entries and associated target variables.

If cross-site data is to be used for training, there is usually a particularly large problem with regard to privacy and data protection, because the data from the different measurement sites must be shared, in other words, given out of hand, so that it can be used at another measurement site or even a third site for training learning models.

This is especially true when it comes to data with medical relevance and/or other particularly sensitive data. Decentralized learning does offer the possibility of greater privacy, as the data does not have to be merged centrally, but can remain at the respective measurement site. However, as already mentioned, decentralized learning generally leads to less accurate models.

The invention offers a great advantage here, because the sharing or merging of data is possible at different levels, whereby the higher the level at which the sharing or merging takes place, the greater the privacy or data protection. However, even the predictions at the lowest level, i.e. the group-specific or group-by-group predictions, contain no or at least only limited private information, as their information content is only related to the prediction, in other words the predicted target. Accordingly, this data can already be shared across measurement locations for the training of ML models while still maintaining privacy.

Cross-site models can be introduced or obtained in various ways in the context of the invention. For example, an intermediate fusion can be performed in which predictions of the lowest level models are shared across sites in order to train an ML model with the pooled data. It is also possible to perform a “late fusion”, where predictions of the next higher level are shared first, resulting in a weighted aggregation/ensemble model. In all cases where the predictions of the models are merged, whether at the lowest or higher levels, harmonization effects are achieved and privacy is increased.

One embodiment of the training method according to the invention is characterized in that, in step A3, separate resampling-based measurement site-specific group learning models are trained and tested for each measurement site with the learning data sets belonging to the respective measurement site, the resampling method including in each case, that at least one training is performed with a part of the measurement site-specific group sub-measurement data sets and associated measurement site-specific target variables, and at least one testing is performed with another part of the measurement site-specific group sub-measurement data sets, and wherein the at least one testing for the respective measurement site provides predictions for different groups, measurement site-specific and group-specific predictions.

Sharing the learning data collections, in other words raw data, is then not necessary. The data from each measurement site can be processed independently of the data from other measurement sites, whereby the grouping according to the invention and the resampling-based training and testing to obtain the group-specific predictions are carried out measurement site by measurement site. As a result, group-specific predictions are available for the individual measurement sites, which are also referred to here as measurement site-specific and group-specific predictions.

It may then be further provided that in step A5 separate measurement site-specific prediction group learning models are trained for each measurement site, in particular without using a resampling method, whereby the training is preferably carried out in each case with all measurement site-specific group sub-measurement data sets and associated measurement site-specific target variables. Measurement site-specific prediction group learning models obtained in this way can be used in particular for subsequent prediction.

In step A4, the measurement site-specific and group-specific predictions and associated measurement site-specific target variables of different measurement sites can then be merged and thus a merging measurement site-specific and group-spanning meta-learning model can be trained, in particular without using a resampling method. This scenario corresponds to the intermediate data fusion already mentioned above.

Furthermore, it may be provided that in step A4 a resampling-based measurement site-specific cross-group meta-learning model is trained and tested for each measurement site, wherein the resampling method includes that at least one training is performed with a part of the measurement site-specific and group-specific predictions and the associated measurement site-specific target variables of the learning data collections, and at least one testing is performed with another part of the measurement site-specific and group-specific predictions and the associated measurement site-specific target variables of the learning data collections, and wherein the at least one testing provides measurement site-specific cross-group predictions.

This has proven to be particularly useful when there is no intermediate data fusion and the measurement site and group-specific predictions from different measurement sites are not shared or merged.

A cross-site meta-learning model can then be trained, preferably without using a resampling method, with the site-specific cross-group predictions and associated measurement site-specific target variables originating from multiple measurement sites, so that it can provide a cross-site and cross-group prediction from multiple site-specific cross-group predictions.

Further preferably, especially in step A4, a measurement site-specific cross-group meta-learning model is then trained for each measurement site, preferably without using a resampling method, whereby the training is carried out with the measurement site-specific and group-specific predictions and the associated measurement site-specific target variables of the respective learning data collection, preferably with all measurement site-specific and group-specific predictions and associated measurement site-specific target variables. The non-resampling-based measurement site-specific cross-group meta-learning models obtained in this way can be used in particular for subsequent prediction.

It can also be provided that
an additional learning data collection is provided by an additional measurement site that is different from the measurement sites,
and a grouping of the measurement data entries of the measurement data sets of the additional learning data collection into several groups as provided for in step A2 is carried out, whereby additional group sub-measurement data sets are obtained,
the additional group sub-measurement data sets and the target variables of the additional learning data collection are each fed to the measurement site-specific prediction group learning models belonging to the measurement sites, trained in particular without using a resampling method, and measurement site-specific and group-specific predictions are obtained from these,
the measurement-site-specific and group-specific predictions are fed to the measurement site-specific cross-group meta-learning models belonging to the measurement sites, which are trained in particular without using a resampling method, and measurement site-specific cross-group predictions are obtained from these models,
with the measurement site-specific cross-group predictions and the target variables of the additional learning data collection, an additional cross-measurement site meta-learning model is trained preferably without using a resampling method.

In other words, it is possible to use trained learning models belonging to different measurement sites in order to make predictions for data from yet another measurement site. By way of example only, learning models are trained with data from measurement site A and learning models are trained with data from measurement site B, thus obtaining measurement site-specific learning models for A and B. Then data from a third measurement site C is provided and the data from C is fed to both learning models belonging to measurement site A and learning models belonging to measurement site B and predictions are obtained. It should be emphasized that the scenario with measuring sites A, B and C is purely exemplary and that a different number of measuring sites is of course also possible.

In an advantageous further development of the training method according to the invention, it is further provided that the trained measurement site-specific cross-group meta-learning model of at least one measurement site is fed with measurement site-specific and group-specific predictions of at least one other measurement site together with associated target variables, thereby obtaining measurement site-specific cross-group cross-predictions. Preferably, all measurement sites and group-specific predictions and associated target variables of the (respective) other measurement site are supplied.

The cross-site meta-learning model can then be trained with the site-specific cross-group predictions and the measurement site-specific cross-group cross-predictions.

In an advantageous further development of the prediction method according to the invention, it is provided that
in step B2, measurement site-specific prediction group learning models of different sites obtained by performing the training method according to the invention, and/or resampling-based measurement site-specific group learning models of different sites obtained by performing the training method according to the invention, and a merging measurement site and group meta-learning model obtained by performing the training method according to the invention, in particular without using a resampling method, are provided,
the group sub-measurement datasets obtained in step B3 are fed to the measurement site-specific prediction group learning models or the resampling-based measurement site-specific group learning models of different measurement sites in step B4 and measurement site-specific and group-specific predictions are obtained,
the predictions of different measurement sites belonging to a group are combined with each other by a statistical method, in particular averaging, so that a group-specific prediction is obtained for each group,
the group-specific predictions are fed into the merging meta-learning model that spans the measurement site and groups, and a prediction is obtained from this model that spans the measurement location and groups.

This approach has proved particularly useful in cases where an intermediate data fusion was carried out during training, in which the predictions of the lowest level models were combined or merged.

Alternatively or additionally, it may be provided that
in step B2, measurement site-specific prediction group learning models of different sites obtained by performing the training method according to the invention, and/or resampling-based measurement site-specific group learning models of different measurement sites obtained by performing the training method according to the invention, and a merging measurement site-and group-spanning meta-learning model obtained by performing the training method according to the invention, in particular without using a resampling method, are provided,
the group sub-measurement data sets obtained in step B3 are fed to the measurement site-specific prediction group learning models or the resampling-based measurement site-specific group learning models n of the different measurement sites in step B4 and measurement site-specific and group-specific predictions are obtained,
the measurement site and group-specific predictions are fed site by site to the merging measurement site and cross-group meta-learning model, thus obtaining measurement site-specific cross-group predictions for each measurement site,
the measurement site-specific cross-group predictions are combined with each other using a statistical procedure, in particular averaging, so that a prediction is obtained for all measurement sites and groups.

This has proven to be another suitable prediction variant, especially in cases where an intermediate data fusion took place during training.

A further advantageous embodiment of the prediction method according to the invention is further characterized in that
in step B2, measurement site-specific prediction group learning models of different sites obtained by performing the training method according to the invention, and/or resampling-based measurement site-specific group learning models of different measurement sites obtained by performing the training method according to the invention and measurement site-specific cross-group meta-learning models, which were obtained by performing the training method according to the invention, in particular without using a resampling method, and a cross-site meta-learning model which was obtained by performing the training method according to the invention, in particular without using a resampling method, and/or an additional cross-site meta-learning model which was obtained by performing the training method according to the invention, in particular without using a resampling method, are provided,
the group sub-measurement data sets obtained in step B3 are fed to the measurement site-specific prediction group learning models or the resampling-based measurement site-specific group learning models of the different measurement sites in step B4 and measurement site-specific and group-specific predictions are obtained,
the measurement site-specifics and group-specific predictions are fed site-by-site to the respective corresponding measurement site-specific cross-group meta-learning model and thus measurement site-specific cross-group predictions (for each measurement site) are obtained,
the measurement site-specific cross-group predictions are fed to the cross-measurement site meta-learning model or the additional cross-measurement site meta-learning model and a cross-measurement site and cross-group prediction is obtained from this.

This embodiment example is particularly suitable for the case that a late data fusion took place during training, in the context of which predictions of the model(s) of a higher than the lowest level were combined or merged.

A further object of the present invention is a computer program comprising instructions which, when executed on at least one computer, cause the at least one computer to perform the steps of the training method according to the invention and/or the steps of the prediction method according to the invention.

It is also an object of the invention to provide a computer-readable medium comprising instructions which, when executed on at least one computer, cause the at least one computer to perform the steps of the training method according to the invention and/or the steps of the prediction method according to the invention.

It is also possible that at least some of the steps of the training method according to the invention and/or at least some of the steps of the prediction method according to the invention are carried out in a cloud. This is to be understood in particular as the use of externally or remotely or distributedly operated services via the Internet (cloud computing), including both “Infrastructure as a Service” (IaaS) and “Platform as a Service” (PaaS) as well as “Software as a Service” (SaaS).

Finally, it is an object of the invention to provide a device for carrying out the training method according to the invention and/or the prediction method according to the invention, comprising a computer with a data memory on which the computer program according to the invention is stored.

In a preferred embodiment, the device according to the invention comprises an MRI scanner device. This is designed and set up to create MRI images of a patient. The device according to the invention has proven to be particularly suitable for locally executing the training method and/or prediction method according to the invention.

With regard to the embodiments of the invention, reference is also made to the subclaims and to the following description of several embodiments with reference to the accompanying drawing.

The drawing shows a purely schematic representation:

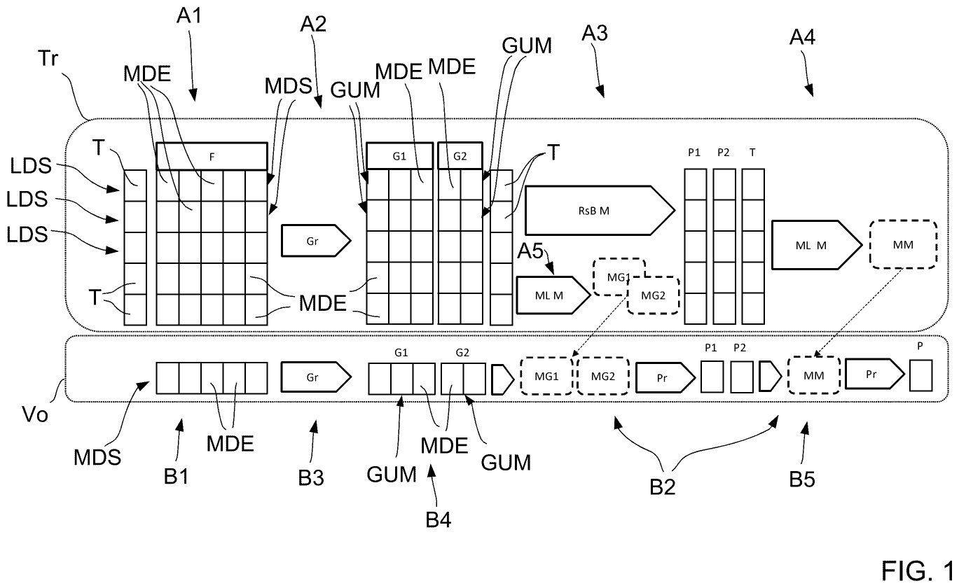

FIG. 1 a first embodiment example of the computer-implemented training method according to the invention for the case where the learning data sets originate from a measurement site, and a first embodiment example of the computer-implemented prediction method according to the invention using trained models obtained with the training method according to this figure;

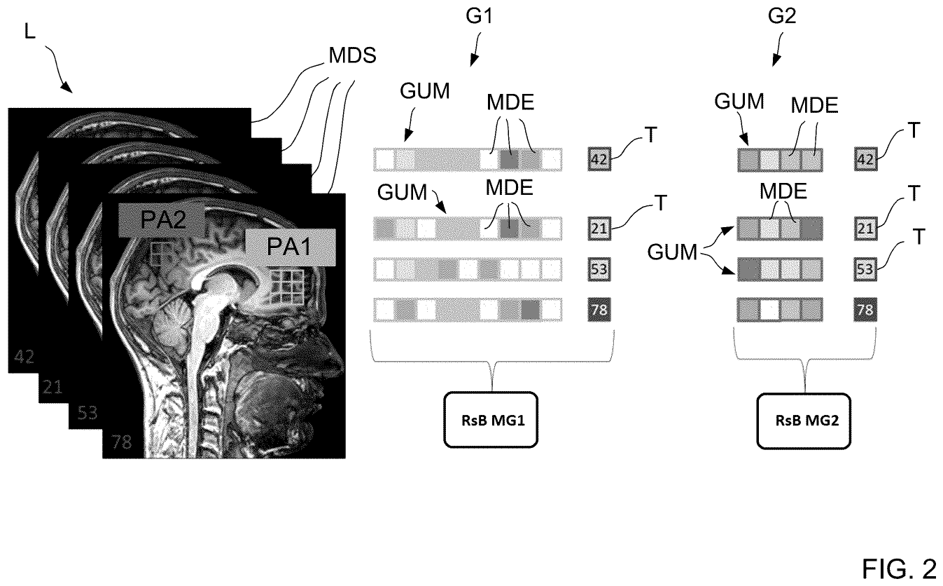

FIG. 2 MRI images of human brains of the learning data collection from FIG. 1 together with a purely schematic representation of the grouping into brain parcels;

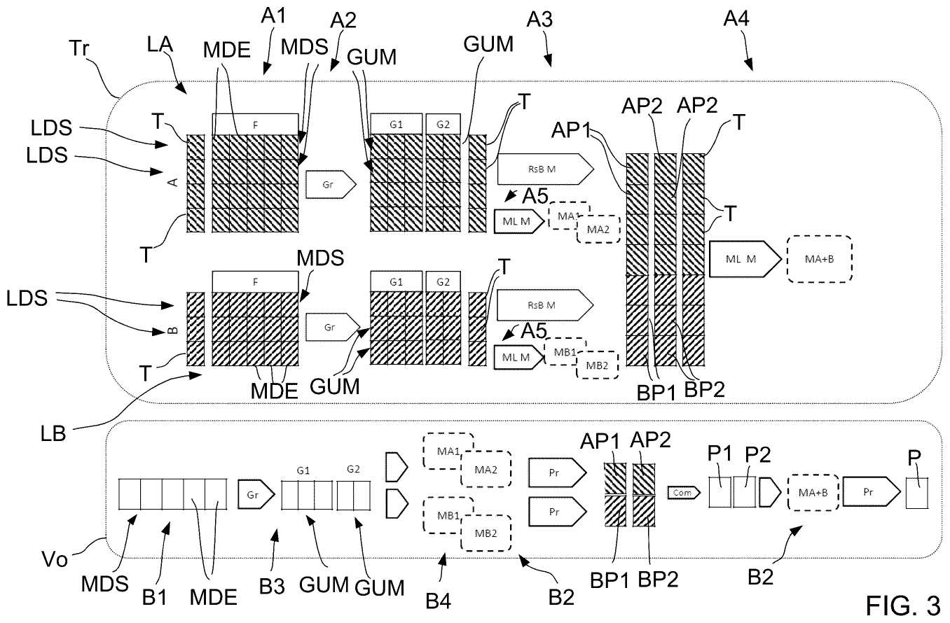

FIG. 3 a second embodiment example of the computer-implemented training method according to the invention for the case where the learning data sets originate from two different measurement locations, and a second embodiment example of the computer-implemented prediction method according to the invention using trained models obtained with the training method according to this figure;

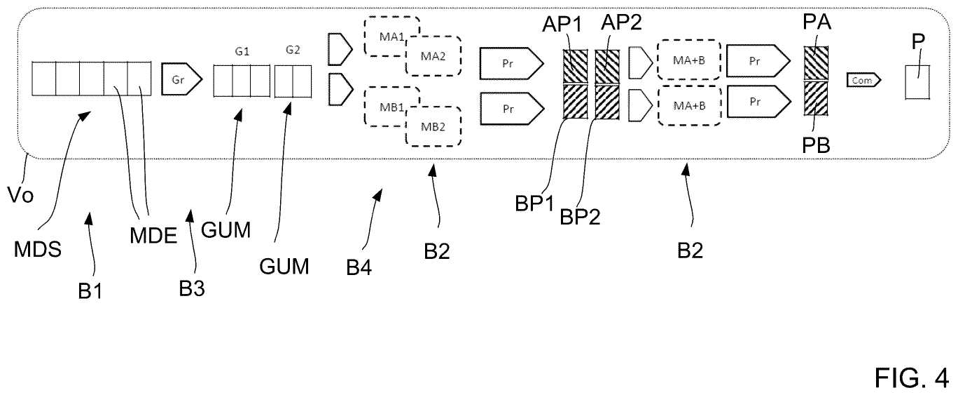

FIG. 4 a third embodiment of the computer-implemented prediction method according to the invention, which also uses trained models obtained with the training method according to FIG. 3;

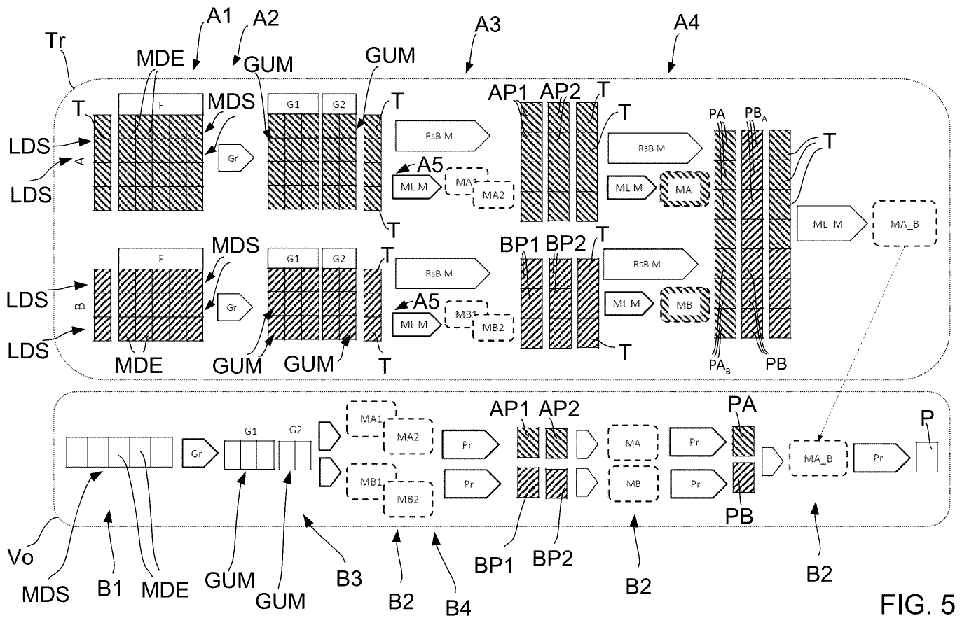

FIG. 5 a third embodiment of the computer-implemented training method according to the invention for the case where the learning data sets originate from two different measurement locations, and a fourth embodiment of the computer-implemented prediction method according to the invention using trained models obtained with the training method according to this figure; and

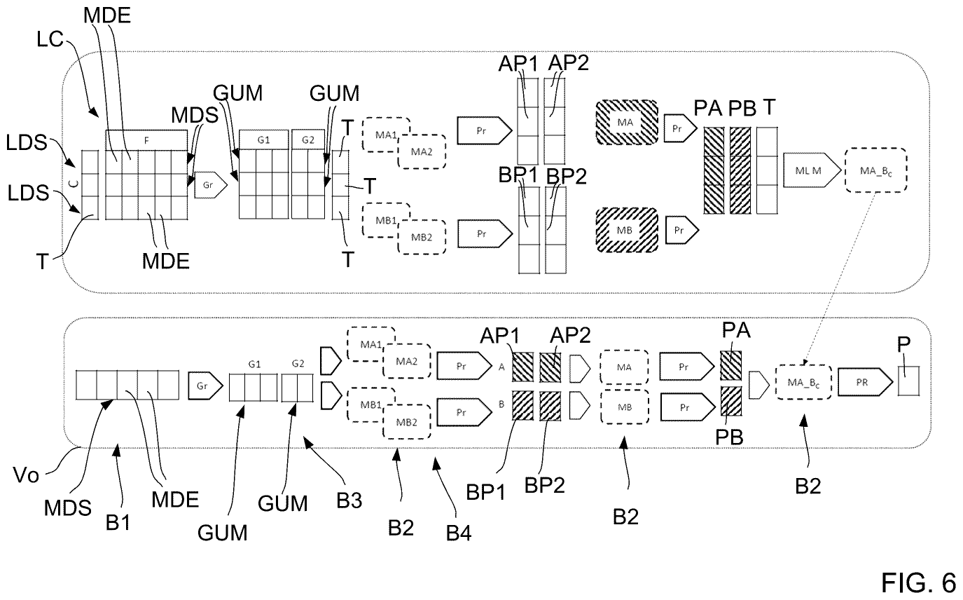

FIG. 6 a fourth embodiment example of the computer-implemented training method according to the invention for the case where the learning data sets originate from three different measurement sites, and a fifth embodiment example of the computer-implemented prediction method according to the invention using trained models obtained with the training method according to this figure.

In the figures, identical or corresponding elements are marked with the same reference signs.

FIG. 1 above shows a purely schematic block diagram of the steps of a first embodiment of the computer-implemented training method Tr according to the invention, which are summarized in a frame designated Tr. Below this is a further frame, labeled Vo, within which the steps of a first corresponding embodiment example of the computer-implemented prediction method according to the invention are shown, which uses machine learning models MG1, MG2, MM obtained with the training method according to FIG. 1. In the figure, the use is also indicated schematically by dashed arrows.

FIGS. 3, 5 and 6 have a structure analogous to FIG. 1, in each case with an embodiment example of a training method according to the invention at the top and an embodiment example of a prediction method according to the invention at the bottom, which uses models from the method above in each case.

In the training method according to FIG. 1, a learning data set L with several learning data sets LDS is provided in a step A1. Each learning data set LDS comprises a measurement data set MDS with several measurement data entries ME and a target variable T assigned to the measurement data set MDS. In the example shown here, all learning data sets LDS have the same structure, in particular the same number of measurement data entries MDE each with an assigned target variable T. The measurement data entries ME can also be referred to as features, which is indicated by the abbreviation F in the figure.

In the example shown here, the learning data sets LDS are MRI images of human brains of different patients of different ages. This is illustrated in FIG. 2, which shows four such images as examples. MRI stands for magnetic resonance imaging. The MRI images were obtained in a sufficiently well-known manner using a magnetic resonance imaging system located, for example, in a hospital in which the patients were examined. The patient ages of 42, 21, 53 and 78 years, for example, are also shown in FIG. 2 for the respective image. The measurement data entries MDE are given by the voxelwise gray matter volume (voxelwise GMV).

In step A2 of the training method according to FIG. 1, the measurement data entries MDE of the measurement data sets MDS of the learning data collection L are grouped Gr into several groups G1, G2. This results in group sub-measurement data sets GUM, whereby the group sub-measurement data sets GUM of a group G1, G2 each comprise corresponding measurement data entries ME of different measurement data sets MDS. For reasons of clarity, this is shown in FIG. 1 as an example for 2 groups G1, G2. It should be emphasized that a grouping into a larger number of groups is of course possible and will generally take place.

In the example shown here, a grouping is made into brain parcels of a brain parcellation scheme, also known as brain atlases. One parcel can be given by the hippocampus, for example, and other parcels by other brain regions. Another brain atlas is, for example, the Schaefer Atlas [Schaefer et al. 2018]. FIG. 2 shows—purely schematically—two parcellations PA1, PA2 in the anterior MRI image. As can be seen, the one plot PA1 on the right in FIG. 2, which corresponds to the first group G1, comprises nine neighbouring voxels and the second plot PA2 on the left in FIG. 2, which corresponds to group G2, comprises four neighbouring voxels, whereby this is also to be understood purely as an example. For the other measurement data sets MDS, i.e. MRI images of other patients of other ages, the grouping is completely analogous. It should be noted that, although a different number of measurement data entries MDE per group G1, G2 is shown in FIG. 1 for reasons of clarity, the principle can be understood in the same way here. It should also be noted that in the purely schematic representation of the learning data set L in FIG. 1, one line corresponds to one patient.

Finally, it should be emphasized that even if the method according to the invention is particularly suitable for MRI images, it can in principle also be applied to any other type of data-other examples of applications include other medical patient data, genetic data, natural language processing and computer vision.

It is also possible, for example, that the various learning data sets LDS belong to different persons and the measurement data sets MDS are given by or comprise images of at least part of the face and/or at least part of the body of the persons, and the target variables T relate to or are given by characteristics of the persons. Then, for example, a grouping into different areas of the face or body can take place.

A further example is that the various learning data sets LDS belong to different sections of the earth's surface and the measurement data sets MDS are given by or comprise image recordings, in particular satellite images, of the earth's surface sections, and the target variables T relate to or are given by properties of the earth's surface sections, in particular the presence of certain elements, for example the presence of fields and/or rivers and/or lakes. It would then be possible, for example, for grouping to take place according to certain areas or parts of the image recordings.

It is also possible that the various learning data sets LDS belong to different persons and the measurement data sets MDS comprise information about the user behavior of the persons, in particular on at least one website, and the target variables T relate to or are provided by information about actions performed by the persons, in particular purchases made by the persons. Then, for example, different areas of the at least one website can form or correspond to different groups, in other words, they can be grouped into different website areas or sections.

As a further example, it may be mentioned that the different learning data sets LDS belong to different DNA sequences and/or protein sequences and/or gene expressions and the measurement data sets MDS are given by or comprise information about the DNA sequences and/or protein sequences and/or gene expressions, in particular information concerning the structure thereof, and the target variables T concern or are given by features of the DNA sequences and/or protein sequences and/or gene expression data, in particular binding sites and/or protein-protein interactions and/or solvent properties thereof. Then, for example, different DNA or gene regions can form or correspond to different groups.

After the grouping into brain parcels according to the invention- or one of the other groupings described above by way of example-has been carried out, a separate machine learning model is trained and tested for each group G1, G2 in step A3 using a resampling method. This is also indicated schematically in FIGS. 1 and 2, in FIG. 1 by the arrow RsB M, where the abbreviation stands for resampling-based modeling. The model RsB MG1 is trained for group G1 and the model RsB MG2 for group G2 (see FIG. 2).

In principle, the user is free to choose which type of learning models are used. The models comprise one or more machine learning algorithms, which can be decision trees and/or support vector machines, for example. Preferably, inherently interpretable models are selected.

The resampling-based method includes that at least one training is performed with a part of the group sub-measurement data sets GUM belonging to the respective group G1, G2 and the associated target variables T, and at least one testing is performed with another part of the group sub-measurement data sets GUM belonging to the respective group G1, G2, and wherein the at least one testing provides predictions for different groups G1, G2, group-specific predictions. In a manner known per se, the data is subdivided once or several times into a training and test part; one can also speak of training and test sets. In the present example for the respective group G1, G2—the data, specifically the group sub-measurement data sets GUMs belonging to the respective group G1, G2 with associated target variables T, of some patients, for example one half of the patients, are used as training sets and the data GUMs of the remaining patients, i.e. the second half, are used as test sets and a cross-validation, for example a leave-one-out cross-validation or k-fold cross-validation, is performed. The groupwise feeding of the training set GUMs and associated target variables T to the group learning models RsB MG1, RsB MG2 is also shown schematically in FIG. 2 on the right.

The test set GUMs are then used to obtain “out-of-sample” predictions P1, P2 (see FIG. 1) for the patient age in groups. Since, as a result of the group-by-group procedure, separate predictions P1, P2 are obtained for each group, these are also referred to as group-specific predictions P1, P2. In other words, the data from the second half is used for training and the data from the first half for testing, so that out-of-sample predictions P1, P2 are also obtained for the first half. With this approach, group-specific predictions P1, P2 can be obtained in a number that corresponds to the number of measurement data sets MDS of the learning data set L and thus to the total number of patients. However, this is not absolutely necessary.

With the group-specific predictions P1, P2 of the patient ages and the known patient ages, i.e. target variables T of the learning data collection L, another machine learning model, cross-group meta-learning model MM, is trained in step A4 without using a resampling method. It can also be said that several, in this example two, levels of machine learning models are trained, whereby the group-wise training can be referred to as Level0 training and the training of the meta-model MM as Level1 training.

In FIG. 1, the training without using a resampling method of the meta-model MM is indicated by an arrow ML M. Following this training, the model MM can then provide a cross-group prediction P from several group-specific predictions P1, P2 assigned to different groups G1, G2.

Furthermore, in step A5, a further machine learning model is trained for each group G1, G2 without using a resampling method with group sub-measurement data sets GMU belonging to the respective group G1, G2 and associated target variables T, prediction group learning model MG1, MG2, whereby the training is carried out here with all group sub-measurement data sets GMU belonging to the respective group G1, G2 and associated target variables T, in other words here with the learning data set data of all patients. In FIG. 1, this training is indicated purely schematically with a further arrow ML M and to the right of this the prediction group learning models MG1, MG2. The training of MG1 and MG2 can of course also take place before the training of the model MM, e.g. before, after or parallel to the resampling-based training of RsB MG1 and RsB MG2.

The resulting trained learning models MM and MG1, MG2 can then be used to estimate the age of other patients based on MRI images of their brains.

As noted above, this is shown purely schematically in FIG. 1 below. It should be noted that training the prediction group learning models MG1, MG2 is optional, as the resampling-based group learning models RsB MG1, RsB MG2 can in principle also be used as an alternative to these for prediction. Additional use is also possible.

In the event that training with measurement data sets MDS with image recordings of at least part of the face and/or at least part of the body of persons and associated target variables with characteristics of the persons has taken place, the age and/or gender of another person, for example, can be predicted based on a new recording.

In the event that training with measurement data sets MDS with image recordings, in particular satellite images, of earth surface sections and associated target variables (T) with properties of the earth surface sections has been carried out, it is possible, for example, to predict for a new recording of a further earth surface section whether this comprises at least one river and/or at least one lake and/or at least one field.

In the event that training was performed with measurement data sets MDS with information about the user behavior of persons and associated target variables T with information about actions performed by the persons, a purchase probability can be predicted, for example, for a measurement data set MDS with information about the user behavior of another person.

In the event that training with measurement data sets MDS with information about DNA sequences and/or protein sequences and/or gene expressions was carried out, at least one binding site or protein-protein interaction can be predicted for a measurement data set MDS, for example.

Specifically, a measurement data set MDS with several measurement data entries MDE is provided for the prediction in step B1. This can also be referred to as test sample features.

In step B2, prediction group learning models MG1, MG2 trained according to step A5 above and a cross-group meta-learning model MM trained according to step A4 above are provided.

The measurement data entries MDE of the provided measurement data set MDS are grouped in step B3 analogous to step A2, whereby a group sub-measurement data set GUM is obtained for each group. Analogous to step A2 in the present case or in the case of training with MRI images means that the new MRI image of the patient of unknown age or the corresponding voxel-wise gray cell volumes are divided into the same brain parcels as is done in the training procedure. It is understood that even if the case of MRI images of brains is discussed below as an example, the procedure can be completely analogous with regard to the other examples mentioned above.

In step B4, the group sub-measurement data sets GUM are each fed to the associated prediction group learning models MG1, MG2 and a group-specific prediction P1, P2 is obtained as output from each of these. As noted, the models RsB MG1, RsB MG2 can also be used alternatively or additionally (not additionally shown in FIG. 1 below for reasons of clarity). In the embodiment example described here, a patient age is obtained as prediction P1, P2 for each group, i.e. each brain cell, whereby the group-specific, i.e. cell-specific, ages may differ from one another.

In step B5, the group-specific predictions for age P1, P2 are fed to the cross-group meta-learning model MM and from this, exactly one cross-group prediction P, in other words a patient age for the whole brain, is obtained as output.

The advantages of this approach include increased prediction accuracy, improved generalizability of model predictions and increased interpretability/explanability both at model level and at data point level. Interpretability at model level means that each trained model also provides out-of-sample predictions and thus predictive accuracy. This prediction accuracy can be interpreted as a measure of importance for the predictive power of the measurement data entries MDE used by this model and thus of the groupings. The interpretability at data point level is given by the fact that the prediction accuracies of each model are checked for—also new—data points in the prediction procedure and can serve as an explanation for differences in the prediction accuracy of the entire prediction procedure for this data point. If the prediction accuracy of models with certain measurement data entries MDE or grouping as input is different than expected, this can provide information as to why the prediction of the entire prediction procedure is different than expected.

The computer-implemented training method according to the invention is particularly suitable for cases where learning data is available from or at different measurement sites. An embodiment example for such a cross-site scenario is shown purely schematically in FIG. 3. In the following, we will look in particular at how the procedure in this case differs from that shown in FIG. 1. With regard to the remaining, identical aspects, reference is made to the previous description.

In contrast to the example in FIG. 1, in the cross-site scenario in step A1, several learning data collections originating from different measurement locations are provided. In the example shown in FIG. 3, specifically two learning data collections LA, LB originating from the measurement sites A, B. It is understood that, purely by way of example and for reasons of clarity, FIG. 3 shows two measurement locations A, B and that there may of course be more.

The different measurement sites A, B are given here by different hospitals, each of which has its own MRI system, by means of which the measurement data sets MDS of the learning data collections LA, LB were obtained for patients of known age. Each of the two learning data collections LA, LB is similar in structure to the learning data collection L from FIG. 1, so that reference is also made to the above description in this respect.

In the cross-site scenario shown in FIG. 3, in step A3, separate resampling-based, site-specific group learning models are trained and tested for each and at each measurement site A, B using the learning data sets LDS belonging to the respective measurement site A, B. This is again indicated by arrows labeled RsB M in the figure. This is again indicated in the figure by arrows labeled RsB M. The resampling method also includes here that at least one training is carried out with a part of the measurement site-specific group sub-measurement data sets GUM belonging to the respective measurement site A, B and associated measurement site-specific target variables T, and at least one testing is carried out with another part of the measurement site-specific group sub-measurement data sets GUM. The testing then provides—for the respective measurement site A, B —predictions for different groups, in other words measurement location-specific and group-specific predictions AP1, AP2, BP1, BP2. In FIG. 3, a hatching tilted to the left is used for measuring site A and a hatching tilted to the right is used for measuring site B to make a better distinction. The predictions AP1, BP1 specific to measuring site A or B and group G1 are correspondingly shaded to the left or right and labeled P1. The same applies to group G2.

As in the example shown in FIG. 1, a cross-validation, such as a leave-one-out cross-validation or k-fold cross-validation, can be performed, with the only proviso that this is also performed separately for the two measurement sites A, B. In the embodiment example shown, a number of out-of-sample predictions is obtained in this way for each measurement sites A, B that corresponds to the number of measurement data sets MDS of the respective learning data collection LA, LB. Again, this is not mandatory.

Furthermore, in step A5, separate measurement location-specific prediction group learning models MA1, MA2, MB1, MB2 are trained for each measurement location without using a resampling method, whereby the training here is carried out with all measurement site-specific group sub-measurement data sets GUM and associated measurement location-specific target variables T in each case. Again, the training of the measurement site-specific prediction group learning models MA1, MA2, MB1, MB2 is optional.

In step A4, the measurement site-specific and group-specific predictions AP1, AP2, BP1, BP2 and associated measurement site-specific target variables T of the two different measurement sites A, B are then merged (“data pooling”) and a non-resampling-based merging measurement site and group-spanning meta-learning model MA+B is trained with this data from both measurement sites A, B. The merging of the measurement sites and group-specific predictions AP1, AP2, BP1, BP2 and associated measurement site-specific target variables T can, for example, take place by measurement site A transferring its predictions AP1, AP2 and target variables T to measurement site B or vice versa. Of course, both measuring sites A and B can also transfer the data to a third party, which then uses them together for training.

Data from different measurement sites are usually heterogeneous. In particular, there are systematic differences between the measurement setups used, such as scanners in the case of MRI images, and the data collection parameters [Chen 2021, Mali et al]. The training method according to FIG. 3 enables harmonization here, since the predictions of measurement site-specific group learning models are based on the target variable itself and not on the measurement site-specific associations between the measurement data entries MDE and the target variable T. For example, if the intensity of each brain parcel is higher for measurement site A than in measurement site B, this would lead to different measurement site-dependent representations using normal data pooling methods, such as the determination of the arithmetic mean. However, the use of the predictions of measurement site-specific group learning models are now always predicted target variable values and thus oriented towards the target variable. They therefore no longer include these differences in intensity. On the other hand, a high degree of privacy is ensured despite the cross-site data, as no sharing of the raw data is required, but only the predictions AP1, AP2, BP1, BP2 of the different measurement sites A, B are merged. This means that collaborations between different measurement sites A, B are enabled without sharing raw data or other proprietary data.

The following then applies to a subsequent prediction (see FIG. 3 below), whereby the differences to the embodiment example of the prediction method according to the invention as shown in FIG. 1 are explained again and reference is also made to its description above.

In step B2, the obtained measurement site-specific prediction group learning models MA1, MA2, MB1, MB2 (alternatively or additionally the measurement site-specific prediction group learning models MA1, MA2, MB1, MB2, which is not additionally shown in FIG. 3 for reasons of clarity) and the obtained merging measurement site-and group-spanning meta-learning model MA+B are provided.

The group sub-measurement data sets GUM of the measurement data set MDS provided in step B1 are fed to the measurement location-specific prediction group learning models MA1, MA2, MB1, MB2 in step B4. The group sub-measurement data sets GUM are fed to both the prediction group learning models MA1, MA2 of measurement site A and the prediction group learning models MB1, MB2 of measurement site B, so that measurement location-and group-specific predictions P1, P2 are obtained for each measurement location A, B (also shaded accordingly). In the present example, a predicted patient age is obtained for each group, i.e. each brain parcel, once using the models MA1, MA2 trained with the data LA from measurement site A and once using the models MB1, MB2 trained with the data LB from measurement site B.

The predictions AP1, AP2, BP1, BP2 of the two different measuring sites A, B, each belonging to a group G1, G2, are combined with each other by a statistical procedure, in this case by forming their mean value, so that exactly one group-specific prediction P1, P2 is obtained for each group across measuring locations. In other words, the mean value of AP1 and BP1 and the mean value of AP2 and BP2 are formed.

The (only) group-specific predictions P1, P2 obtained by averaging are fed to the non-resampling-based merging measurement and cross-group meta-learning model MA+B and from this a measurement and cross-group prediction P—in the present example again a patient age—is obtained.

FIG. 4 shows a further example of a prediction method according to the invention that uses learning models obtained according to the training method in FIG. 3, i.e. using cross-site data. As can be seen, the difference is that no mean value is formed from the measurement site-specific and group-specific predictions AP1, AP2, BP1, BP2, but these are fed site by site to the merging measurement site and cross-group meta-learning model MA+B, so that measurement site-specific cross-group predictions PA, PB are obtained. These are then combined with each other using a statistical procedure, in particular averaging, so that a prediction P is obtained from PA and PB across measurement sites and groups.

FIG. 5 shows a further example of a computer-implemented training method according to the invention for the cross-site case. The main difference to the example shown in FIG. 3 is that data from the two different measurement sites A, B are merged at a later point in time. One can also speak of a “late data fusion”, while the example in FIG. 3 corresponds to an “intermediate data fusion” already of the predictions of Level0.

The scenario in FIG. 5 above corresponds to that in FIG. 3 until the measurement location and group-specific predictions AP1, AP2, BP1, BP2 are received. However, these are not merged, but processed separately, e.g. further at the respective measurement site A, B.

In step A4, a resampling-based measurement site-specific cross-group meta-learning model is then trained and tested for each measurement site, where the resampling method includes training with at least a portion of the measurement site-specific and group-specific predictions AP1, AP2, BP1, BP2 and the associated measurement site-specific target variables T of the learning data collections LA, LB, and at least one testing is performed with another part of the measurement site-specific and group-specific predictions AP1, AP2, BP1, BP2 and the associated measurement site-specific target variables T of the learning data collections LA, LB, and wherein the at least one testing provides measurement site-specific cross-group predictions PA, PB.

Furthermore, in particular also in step A4, for each measurement site A, B, a non-resampling-based measurement site-specific cross-group meta-learning model MA, MB is trained, the training being carried out in each case with the measurement site-specific and group-specific predictions AP1, AP2, BP1, BP2 and the associated measurement site-specific target variables T of the respective learning data collection LA, LB, preferably with all measurement site-specific and group-specific predictions AP1, AP2, BP1, BP2 and associated measurement site-specific target variables T.

The trained non-resampling-based measurement site-specific cross-group meta-learning models MA, MB are fed measurement site-specific and group-specific predictions of the respective other measurement site A, B together with associated target variables T and thereby obtain measurement site-specific cross-group cross-predictions PAB, PBA. In other words, the predictions BP1 and BP2 are fed to model MA and the predictions PAB are obtained, and the predictions AP1 and AP2 are fed to model MB and the predictions PBA are obtained. The predictions PAB are accordingly those obtained using a model belonging to measurement site A and data from measurement site B and vice versa for PBA.

A non-resampling-based cross-site meta-learning model MA_B is then trained with the measurement site-specific cross-group predictions PA, PB measurement and the site-specific cross-group cross-predictions PAB, PBA and the corresponding target variables T of the learning data collections LA, LB, so that it can provide a cross-site and cross-group prediction P from several measurement site-specific cross-group predictions PA, PB.

An associated embodiment example of a computer-implemented prediction method according to the invention is shown in FIG. 5 below. This corresponds to those shown in FIGS. 3 and 4 until the measurement-location and group-specific predictions AP1, AP2, BP1, BP2 are obtained. Then, however, the non-resampling-based measurement site-specific cross-group meta-learning models MA, MB are used. Specifically, the group-specific predictions of the associated measurement location A, B, i.e. the predictions AP1 and AP2 are fed to the trained model MA and the boron predictions BP1 and BP2 are fed to the model MB. From the model MA, the cross-group measurement site-specific prediction PA is obtained and from the model MB the cross-group measurement site-specific prediction PB is obtained (one patient age from each measurement site-specific model). These predictions PA, PB are then fed to the non-resampling-based cross-site meta-learning model MA_B, which provides the prediction P, in this case a patient age.

FIG. 6 shows another example of a cross-site scenario. In this case, an additional learning data collection LC from an additional measuring site C different from the measuring sites A and B is used. It should be noted that in addition to the steps according to FIG. 6, steps of the training procedure from FIG. 5 must be completed, namely at least those for obtaining the models MA1, MA2, MB1, MB2 as well as MA and MB, in each case using the corresponding learning data collections LA and LB.

As can be seen in FIG. 6, the measurement data entries MDE of the measurement data sets MDS of the additional learning data collection LC are then grouped into several groups G1, G2 as provided in step A2, whereby additional group sub-measurement data sets GUM are obtained.

The additional group sub-measurement datasets GUM and the target variables T of the additional learning dataset LC are then fed to the trained non-resampling-based measurement location-specific group learning models MA1, MA2, MB1, MB2 belonging to the measurement sites A and B, respectively, and measurement site-specification and group-specific predictions are obtained from these, namely AP1, AP2 on the one hand and BP1, BP2 on the other.

The measurement-site-specific and group-specific predictions AP1, AP2, BP1, BP2 are each fed to the trained non-resampling-based measurement site-specific cross-group meta-learning models MA, MB belonging to the measurement sites A, B and obtained from these measurement site-specific cross-group predictions PA, PB. This is to a certain extent analogous to FIG. 5, but with the proviso that AP1, AP2, BP1, BP2 are obtained with the models MA1, MA2, MB1, MB2, but not using data from these measurement sites A, B, but from the independent measurement site C.

A non-resampling-based additional cross-site meta-learning model MA_Bc is then trained with the measurement site-specific cross-group predictions PA, PB and the corresponding target variables T of the additional learning data collection LC of site C.

As can be seen in FIG. 6 below, a corresponding embodiment of an associated computer-implemented prediction method is the same as the example in FIG. 5, with the only proviso that the non-resampling-based auxiliary cross-measure meta-learning model MA_Bc is used to obtain P. Such a system offers many advantages for federation learning based collaborations of multiple workgroups. As an example, consider a scenario where measurement site/hospital A and measurement site/hospital B use the same training procedure internally to then share predictions on measurement site C with each other or a third party, e.g. researchers. This means that neither the trained models nor the raw data need to be shared with each other. This allows maximum protection for the security and privacy of the data from measurement sites A and B. Measurement site C can be a data set that is accessible to all parties or even publicly. This means that both private and publicly or otherwise shared, large and small data sets can be ideally utilized. An example of such a now more usable large-scale dataset is the UK Biobank (UKB) [Sudlow et al. 2015]. Of course, this exact scenario is just one example and the use of a different number of both private and shared datasets from different sites is possible.

The steps of the above-described embodiments of both training and prediction methods according to the invention can each be carried out by means of at least one computer. One or more computer programs with program code means can be used which, when executed on at least one computer, cause the at least one computer to perform the above steps. The use of a (private and/or public) cloud is also possible.

### REFERENCES

Chen et al. 2021: Chen, J., Müller, V.I., Dukart, J., Hoffstaedter, F., Baker, J.T., Holmes, A.J., Vatansever, D., Nickl-Jockschat, T., Liu, X., Derntl, B., et al. (2021). Intrinsic Connectivity Patterns of Task-Defined Brain Networks Allow Individual Prediction of Cognitive Symptom Dimension of Schizophrenia and Are Linked to Molecular Architecture. Biol. psychiatry 89, 308-319.
Finn et al. 2015: Finn, E.S., Shen, X., Scheinost, D., Rosenberg, M.D., Huang, J., Chun, M.M., Papademetris, X., and Constable, R.T. (2015). Functional connectome fingerprinting: identifying individuals using patterns of brain connectivity. Nat. Neurosci. 18, 1664-1671
Hedderich & Eickhoff 2021: Hedderich, D.M., and Eickhoff, S.B. (2021). Machine learning for psychiatry: getting doctors at the black box? Mol. Psychiatry 26, 23-25
Kaissis et al. 2020: Kaissis, G.A., Makowski, M.R., Rückert, D., and Braren, R.F. (2020). Secure, privacy-preserving and federated machine learning in medical imaging. Nat. mach. Intell
Larabi et al. 2021: Larabi, D.I., Gell, M., Amico, E., Eickhoff, S.B., and Patil, K.R. (2021). Highly accurate local functional fingerprints and their stability. BioRxiv
Mali et al 2021: Mali, S.A., Ibrahim, A., Woodruff, H.C., Andrearczyk, V., Müller, H., Primakov, S., Salahuddin, Z., Chatterjee, A., and Lambin, P. (2021). Making Radiomics More Reproducible across Scanner and Imaging Protocol Variations: A Review of Harmonization Methods. J. Pers. Med. 11
Myszczynska et al. 2020: Myszczynska, M.A., Ojamies, P.N., Lacoste, A.M.B., Neil, D., Saffari, A., Mead, R., Hautbergue, G.M., Holbrook, J.D., and Ferraiuolo, L. (2020). Applications of machine learning to diagnosis and treatment of neurodegenerative diseases. Nat. rev. neurol. 16, 440-456
Schaefer et al. 2018: Schaefer, A., Kong, R., Gordon, E.M., Laumann, T.O., Zuo, X.-N., Holmes, A.J., Eickhoff, S.B., and Yeo, B.T.T. (2018). Local-Global Parcellation of the Human Cerebral Cortex from Intrinsic Functional Connectivity MRI. Cereb. Cortex 28, 3095-3114
Sudlow C et al. 2015: Sudlow, C., Gallacher, J., Allen, N., Beral, V., Burton, P., Danesh, J., Downey, P., Elliott, P., Green, J., Landray, M., et al. (2015). UK Biobank: an open access resource for identifying the causes of a wide range of complex diseases of middle and old age. PLOS Med. 12, e1001779
Yeo et al. 2011: Yeo, B.T.T., Krienen, F.M., Sepulcre, J., Sabuncu, M.R., Lashkari, D., Hollinshead, M., Roffman, J.L., Smoller, J.W., Zöllei, L., Polimeni, J.R., et al. (2011). The organization of the human cerebral cortex estimated by intrinsic functional connectivity. J. Neurophysiol. 106, 1125-1165

1-25. (canceled)

26. Computer-implemented training method for training machine learning models in which
A1) at least one learning data collection (L, LA, LB, LC) with several learning data sets (LDS) is provided, wherein each learning data set (LDS) comprises a measurement data set (MDS) with several measurement data entries (MDE) and a target variable (T) assigned to the measurement data set (MDS), preferably wherein all learning data sets (LDS) have the same structure,
wherein
A2) at least some of the measurement data entries (MDE) of the measurement data sets (MDS) of the at least one learning data collection (L, LA, LB, LC) are grouped into a plurality of groups (G1, G2), whereby group sub-measurement data sets are obtained, the group sub-measurement data set (GUM) of a group (G1, G2) each comprising measurement data entries (MDE) of different measurement data sets (MDS) which correspond to one another,
A3) for each group (G1, G2) a separate machine learning model is trained and tested using a resampling method, resampling-based group learning model (RsB MG1, RsB MG2), wherein the resampling-based method includes that at least one training is performed with a part of the group sub-measurement data sets (GUM) belonging to the respective group (G1, G2) and the associated target variables (T), and at least one testing is performed with another part of the group sub-measurement data sets (GUM) belonging to the respective group (G1, G2), and wherein the at least one testing provides predictions for different groups, group-specific predictions (P1, P2, AP1, AP2, BP1, BP2),
A4) with the group-specific predictions (P1, P2, AP1, AP2, BP1, BP2) and target variables (T) of the at least one learning data collection (L, LA, LB, LC) at least one further machine learning model, cross-group meta-learning model (MM, MA+B, MA_B), is trained in particular without using a resampling method, so that it can provide a cross-group prediction (P) from several group-specific predictions (P1, P2, AP1, AP2, BP1, BP2) assigned to different groups (G1, G2),
A5) optionally for each group (G1, G2) a further machine learning model is trained with group sub-measurement data sets (GUM) belonging to the respective group (G1, G2) and associated target variables (T), in particular without using a resampling method, prediction group learning model (MG1, MG2), the training preferably being performed with all group sub-measurement data sets (GUM) belonging to the respective group (G1, G2) and associated target variables (T).

27. Computer-implemented training method according to claim 26, wherein the different learning data sets (LDS) belong to different patients and the learning data sets (LDS) comprise medically relevant measurement data sets (MDS), in particular measurement data sets (MDS) obtained by means of medical diagnostic procedures, and the target variables (T) relate to or are given by characteristics of the patients, in particular an age and/or a disease state of the patients.

28. Computer-implemented training method according to claim 27, wherein the measurement data sets (MDS) are given by or comprise image recordings, in particular MRI image recordings, of human brains or parts thereof, and the measurement data entries (MDE) each correspond to or are assigned to a voxel, and in that a grouping into brain parcellations takes place.

29. Computer-implemented training method according to claim 26, wherein the different learning data sets (LDS) belong to different persons and the measurement data sets (MDS) are given by or comprise image recordings of at least part of the face and/or at least part of the body of the persons, and the target variables (T) concern or are given by characteristics of the persons, preferably wherein the measurement data entries (MDE) each correspond to or are associated with a pixel, and/or wherein the characteristics of the persons do not concern or are not given by a disease state of the persons.

30. Computer-implemented training method according to claim 26, wherein the different learning data sets (LDS) belong to different sections of the earth's surface and the measurement data sets (MDS) are given by or comprise image recordings, in particular satellite recordings, of the earth's surface sections, and the target variables (T) relate to or are given by properties of the earth surface sections, in particular the presence of certain elements, preferably the presence of fields and/or rivers and/or lakes.

31. Computer-implemented training method according to claim 26, wherein the different learning data sets (LDS) belong to different persons and the measurement data sets (MDS) are given by or comprise information about the user behaviour of the persons, in particular on at least one website, and the target variables (T) relate to or are given by information about actions taken by the persons, in particular purchases made by the persons.

32. The computer-implemented training method according to claim 26, wherein the different learning data sets (LDS) belong to different DNA sequences and/or protein sequences and/or gene expressions and the measurement data sets (MDS) are given by or comprise information about the DNA sequences and/or protein sequences and/or gene expressions, in particular information relating to the structure of these, in particular by the structure of information relating thereto, and the target variables (T) relate to or are given by features of the DNA sequences and/or protein sequences and/or gene expression data, in particular binding sites and/or protein-protein interactions and/or solvent properties thereof.

33. Computer-implemented training method according to claim 26, wherein in step A2 a grouping of the measurement data sets (MDS) predefined by a user or a grouping derived from the at least one learning data collection (L, LA, LB, LC), in particular the structure of the measurement data sets (MDS), preferably obtained by clustering, is carried out.

34. Computer-implemented training method according to claim 26, wherein in step Al learning data collections (L, LA, LB LC) are provided from and in particular at different measurement locations (A, B, C), the learning data collections (L, LA, LB LC) each comprising a plurality of learning data sets (LDS) with measurement data entries (MDE) and associated target variables (T).

35. Computer-implemented training method according to claim 34, wherein in step A3 for each measurement site (A, B, C) separate resampling-based measurement location-specific group learning models are trained and tested with the learning data sets (LDS) belonging to the respective measurement site (A, B, C), the resampling method including in each case, that at least one training is performed with a part of the measurement site-specific group sub-measurement data sets (GUM) and associated measurement site-specific target variables (T), and at least one testing is performed with another part of the measurement site-specific group sub-measurement data sets (GUM), and wherein the at least one testing for the respective measurement site (A, B, C) provides predictions for different groups, measurement site-specific and group-specific predictions (AP1, AP2, BP1, BP2).

36. Computer-implemented training method according to claim 34, wherein in step A5 for each measurement site (A, B, C) separate measurement site-specific prediction group learning models (MA1, MA2, MB1, MB2) are trained, in particular without using a resampling method, the training preferably taking place in each case with all measurement location-specific group sub-measurement data sets (GUM) and associated measurement site-specific target variables (T).

37. Computer-implemented training method according to claim 34, wherein in step A4 the measurement site-specific and group-specific predictions (AP1, AP2, BP1, BP2) and associated measurement site-specific target variables (T) of different measurement locations (A, B, C) are merged and thus a merging measurement site-and group-spanning meta-learning model (MA+B) is trained, in particular without using a resampling method.

38. Computer-implemented training method according to claim 34, wherein in step A4 for each measurement site (A, B, C) a resampling-based measurement site-specific cross-group meta-learning model is trained and tested, wherein the resampling method includes that at least one training is performed with a part of the measurement site-specific and group-specific predictions (AP1, AP2, BP1, BP2) and the associated measurement site-specific target variables (T) of the learning data collections (L, LA, LB, LC), and at least one testing with another part of the measurement site-specific and group-specific predictions (AP1, AP2, BP1, BP2) and the associated measurement-site-specific target variables (T) of the learning data collections (L, LA, LB, LC), and wherein the at least one test provides measurement-site-specific cross-group predictions (PA, PB).

39. Computer-implemented training method according to claim 38, wherein a cross-measurement site meta-learning model (MA_B) is trained with the measurement site-specific cross-group predictions (PA, PB) originating from several measurement sites (A, B, C) and associated measurement site-specific target variables (T), in particular without using a resampling method, so that it can provide a measurement site and cross-group prediction (P) from several measurement site-specific cross-group predictions (PA, PB).

40. Computer-implemented training method according to claim 34, wherein, in particular in step A4, a measurement site-specific cross-group meta-learning model (MA, MB) is trained for each measurement site, in particular without using a resampling method, wherein the training is carried out in each case with the measurement site-specific and group-specific predictions (AP1, AP2, BP1, BP2) and the associated measurement site-specific target variables (T) of the respective learning data collection (L, LA, LB, LC), preferably in each case with all measurement site-specific and group-specific predictions (AP1, AP2, BP1, BP2) and associated measurement site-specific target variables (T).

41. Computer-implemented training method according to claim 36, wherein
an additional learning data collection (LC) is provided by an additional measurement site (C) that is different from the measurement sites (A, B),
and a grouping of the measurement data entries (MDE) of the measurement data sets (MDS) of the additional learning data collection (LC) into several groups (G1, G2) is carried out as provided for in step A2, whereby additional group sub-measurement data sets (GUM) are obtained,
the additional group sub-measurement data sets (GUM) and the target variables (T) of the additional learning data collection (LC) are each fed to the measurement site-specific prediction group learning models (MA1, MA2, MB1, MB2) belonging to the measurement sites (A, B), trained in particular without using a resampling method, and measurement site-specific and group-specific predictions (AP1, AP2, BP1, BP2) are obtained from these,
the measurement site-specific and group-specific predictions (AP1, AP2, BP1, BP2) are fed to the measurement site-specific cross-group meta-learning models (MA, MB) belonging to the measurement sites (A, B), trained in particular without using a resampling method, and measurement site-specific cross-group predictions (PA, PB) are obtained from these,
an additional cross-measurement site meta-learning model MA_Bc is trained with the measurement site-specific cross-group predictions (PA, PB) and the target variables (T) of the additional learning data collection (LC), in particular without using a resampling method.

42. Computer-implemented training method according to claim 40, wherein the trained measurement site-specific cross-group meta-learning model (MA, MB) of at least one measurement site (A, B, C) is supplied with measurement-site-specific and group-specific predictions (AP1, AP2, BP1, BP2) of at least one other measurement site (A, B, C) and measurement-site-specific cross-group cross-predictions (PAB, PBA ) are thereby obtained.

43. Computer-implemented training method according to claim 39, wherein the cross-measurement-site meta-learning model MA_Bc is trained with the measurement site-specific cross-group predictions (PA, PB) and the measurement site-specific cross-group cross-predictions (PAB, PBA).

44. A computer-implemented prediction method for predicting a property using machine learning models obtained by performing the training method according to claim 26, wherein
B1)a measurement data set (MDS) with several measurement data entries (MDE) is provided,
B2)prediction group learning models (MG1, MG2, MA1, MA2, MB1, MB2) trained according to step A5 and/or resampling-based group learning models (RsB MG1, RsB MG2) trained according to step A2 and at least one cross-group meta-learning model (MM, MA+B, MA_B) trained according to step A4, in particular without using a resampling method, are provided,
B3)the measurement data entries (MDE) of the measurement data set (MDS) provided in step B1 are grouped analogously to step A2, whereby a group sub-measurement data set (GUM) is obtained for each group (G1, G2),
B4)the group sub-measurement data sets (GUM) are each fed to the associated prediction group learning models (MG1, MG2, MA1, MA2, MB1, MB2) or the associated resampling-based group learning models (RsB MG1, RsB MG2) and a group-specific prediction (P1, P2) is obtained as output from each of these,
B5)the group-specific predictions (P1, P2, AP1, AP2, BP1, BP2) are fed to the at least one cross-group meta-learning model (MM, MA+B, MA_B) trained in particular without using a resampling method and a cross-group prediction (P) is obtained as output from this model.

45. The computer-implemented prediction method according to claim 44, wherein
in step B2, measurement site-specific prediction group learning models (MA1, MA2, MB1, MB2) of different sites (A, B, C) obtained by performing the training method according to claim 36, and/or resampling-based measurement site-specific group learning models of different sites (A, B, C), obtained by performing the training method according to claim 35, and a merging meta-learning model (MA+B) across measurement locations and groups obtained by performing the training method according to claim 37, in particular without using a resampling method, are provided,
the group sub-measurement datasets (GUM) obtained in step B3 are fed to the measurement site-specific prediction group learning models (MA1, MA2, MB1, MB2) or the resampling-based measurement site-specific group learning models of the different measurement sites (A, B, C) in step B4 and measurement site-and group-specific predictions (AP1, AP2, BP1, BP2) are obtained,
the predictions (AP1, AP2, BP1, BP2) of different measurement sites (A, B, C) belonging to a group (G1, G2) are combined with each other by a statistical method, in particular averaging, so that a group-specific prediction (P1, P2) is obtained for each group (G1, G2),
the group-specific predictions (P1, P2) are fed to the merging meta-learning model (MA+B), which is used to obtain a prediction (P) across all measurement sites and groups.

46. The computer-implemented prediction method according to claim 44, wherein
in step B2, measurement site-specific prediction group learning models (MA1, MA2, MB1, MB2) of different sites (A, B, C) obtained by performing the training method according to claim 36 and/or resampling-based measurement site-specific group learning models of different sites (A, B, C), obtained by performing the training method according to claim 35, and a merging meta-learning model (MA+B) across measurement locations and groups obtained by performing the training method according to claim 37, in particular without using a resampling method, are provided,
in group sub-measurement data sets (GUM) obtained in step B3 are fed to the measurement site-specific prediction group learning models (MA1, MA2, MB1, MB2) or the resampling-based measurement site-specific group learning models of the different measurement sites (A, B, C) in step B4 and measurement site-specific and group-specific predictions (AP1, AP2, BP1, BP2) are obtained,
the measurement site-specific and group-specific predictions (AP1, AP2, BP1, BP2) are fed site by site to the merging measurement site and cross-group meta-learning model (MA+B) and thus measurement site-specific cross-group predictions (PA, PB) are obtained for each measurement site (A, B, C),
the measurement side-specific cross-group predictions (PA, PB) are combined with each other using a statistical method, in particular averaging, so that a prediction (P) is obtained across measurement sites and groups.

47. The computer-implemented prediction method according to claim 44, wherein
in step B2, measurement site-specific prediction group learning models (MA1, MA2, MB1, MB2) of different sites (A, B, C) obtained by performing the training method according to claim 36, and/or resampling-based measurement site-specific group learning models of different sites (A, B, C) obtained by performing the training method according to claim 35, and measurement site-specific cross-group meta-learning models (MA, MB), which were obtained by performing the training method according to claim 40, in particular without using a resampling method, and a cross-site meta-learning model (MA_B), which was obtained by performing the training method according to claim 39, in particular without using a resampling method, and/or an additional cross-site meta-learning model (MA_Bc ), which was obtained by performing the training method according to claim 16, in particular without using a resampling method, are provided,
the group sub-measurement data sets (GUM) obtained in step B3 are fed to the measurement site-specific prediction group learning models (MA1, MA2, MB1, MB2) of the different measurement sites (A, B, C) in step B4 and measurement site-specific and group-specific predictions (AP1, AP2, BP1, BP2) are obtained,
the measurement site-specific and group-specific predictions (AP1, AP2, BP1, BP2) are fed site by site to the respective corresponding measurement site-specific cross-group meta-learning model (MA, MB) and thus measurement site-specific cross-group predictions (PA, PB) are obtained for each measurement location (A, B, C),
the measurement site-specific cross-group predictions (PA, PB) are fed to the cross-measurement location meta-learning model (MA_B) or the additional cross-measurement location meta-learning model (MA_Bc) and a cross-measurement location and cross-group prediction (P) is obtained from this.
